(19) United States
(12) Patent Application Publication
(10) Pub. No.: US 2009/0233348 A1
(43) Pub. Date: Sep. 17, 2009

(54) METHOD FOR ISOLATING PROTEINS FROM PRODUCTION CELLS

(75) Inventors: Thomas Danner, Erpolzheim (DE); Tillman Faust, Weisenheim (DE); Ulrike Richter, Jersey City, NJ (US); Thomas Subkowski, Ladenburg (DE); Marvin Karos, Schwetzingen (DE)

Correspondence Address: CONNOLLY BOVE LODGE & HUTZ, LLP, P O BOX 2207, WILMINGTON, DE, 19899, US

(73) Assignee: BASF SE, LUDWIGSHAFEN (DE)

(21) Appl. No.: 12/377,186

(22) PCT Filed: Aug. 6, 2007
(86) PCT No.: PCT/EP2007/058102

§ 371 Date: Feb. 11, 2009

(30) Foreign Application Priority Data
Aug. 15, 2006 (EP) 06118941.1

(51) Int. Cl. C12N 1/06; C07K 14/39

(52) U.S. Cl. 435/259; 530/427

(57) ABSTRACT

A method for the disruption of biological cells by means of a homogenizer device which

a) comprises an orifice plate having at least one inlet nozzle and an orifice plate having at least one exit nozzle, wherein, in the intermediate space between the orifice plates, a static mixer is situated and, if appropriate, mechanical energy is additionally introduced or
b) comprises an orifice plate having at least one inlet nozzle and a baffle plate, wherein, in the intermediate space between the orifice plate and the baffle plate, if appropriate a static mixer is situated and/or mechanical energy is introduced.

The invention relates to a method of cell digestion of biological cells and the subsequent isolation of proteins from production cells, and also to a device therefor.

### DESCRIPTION OF THE PRIOR ART

If a product produced by fermentation is intracellular, the cell, after the fermentation is complete, must be disrupted (enzyme or protein release). This concerns opening the cells and releasing the internal cell components, especially the sought-after molecule, usually a protein, into the culture broth. It is of no importance in this case whether these proteins are already in the desired native form or are present in inactive form as inclusion bodies. In addition to the sought-after protein, other soluble proteins are also released in this process. The product, after the cell disruption, can then be separated off from what is termed the cell debris, for example by sedimentation in a centrifuge, by filtration or by fractional sedimentation and, if appropriate, further purified.

The physical forces which are used for cell disruption are mechanical forces which can be applied by impact, friction, tension, pressure, pressing, cavitation or sound. In the machines and apparatuses constructed therefor, generally a plurality of these active forces are generated. The externally visible feature is the specific power input. The most effective use possible of the energy introduced with respect to comminution action is in this case an important decision-making criterion for the method to be selected (Storhas W. Bioverfahrensentwicklung [Bioengineering development]. Wiley-VCh Verlag GmbH & Co KgaA Weinheim. 2003). In addition to the purely mechanical cell disruption methods, use can also be made of chemical (e.g. acid/alkali solution, salts, solvents), biological, such as, e.g., enzymes, phages, autolysis (Wisemen, Process Biochem. 4 63-65 (1969); Asenjo J A, Andrews B A, Hunter J B, LeCorre S, Process Biochem. 20: 158-164. 1985, Lam K S, GrootWassink J W D, Enzyme Microb Technol. 239-242. 1985; Tanny G B, Mirelman D, Pistole T. Appl Environ Microbiol 40. 269-273. 1980), thermal and physical (e.g. osmotic pressure, freezing and thawing, freeze drying) or a combination of these methods for cell disruption. For example, Bailey et al. (Improved homogenization of recombinant E. coli following pre-treatment with guanidine hydrochloride. Biotechnol Prog 11: 533-539. 1995) report an improvement of the degree of cell disruption by addition of detergents and chaotropes before the homogenization.

High-pressure homogenization is the disintegration apparatus most frequently used in industrial workup practice. The principle of the high-pressure homogenizer is based on cavitation caused by spontaneous pressure reduction which, with strong tension forces, leads to cell destruction. In the breakdown of the cavitation vapor bubbles, pressures are produced of up to 10⁵ bar which are ultimately also responsible for the destruction of the cell. The suspension is usually fed at a low initial pressure to the piston pump which pressurizes it to the homogenization pressure. In the homogenization unit, the valve converts this pressure into velocity, shearing, normal forces and tension forces. A highly cavitating flow is formed. These processes last, depending on the pressure, approximately 200 to 250 msec. The main factors influencing disintegration in the high-pressure homogenizer are theoretically the homogenization pressure difference, number of passages, design of the homogenization valve, feed concentration and temperature (Storhas, 2003).

During the high-pressure cell disruption, the temperature elevation generally increases in proportion to the pressure difference used (approx. 2.2° C. per 10 Mpa, Storhas, 2003). An increase in product yield due to improved cell disruption at higher pressure differences may be counteracted by heat inactivation of the product. An increase in the pressure difference to improve the degree of cell disruption has as a consequence an increased cooling capacity.

By optimizing the valve construction, energy savings can be achieved with constant product quality and/or improved properties for downstreaming, owing to, e.g., smaller cell debris or narrower size distribution of the cell fragments for the same energy inputs. For example, Storhas (2003) demonstrates that a knife edge valve, in experiments on the release of enzymes from baker's yeasts, has the highest efficiency, followed by a conical valve and flat valve.

A commercially available variant of high-pressure cell disruption is the “Microfluidizer” (from Microfluidics, USA). In this case the cell suspension is brought to the desired pressure at a constant flow rate by using an intensifier pump. The cell suspension is then passed through exactly defined microchannels having a fixed geometry within what is termed the disruption chamber, as a result of which the cell suspension is accelerated to high velocities. Very high shear rates are generated hereby (=shear zone). Subsequently to the shear zone the cell suspension is passed into an impact zone in which the cells are disrupted by impact. For this disruption method, a disruption degree of up to 99% for a single passage of an E. coli suspension at 1240 bar may be expected (Microfluidics brochure, Innovation by Microfluidizer processor technology, 2005).

Frequently, cells are also disrupted by impact-pressure comminution in stirred ball mills (SBM), wherein here predominantly large cell debris is formed. The cell disruption by use of rotor-stator machines and colloid mills is conceivable.

The size of the cell debris resulting from mechanical cell disruption can directly affect the separation result in a disk separator; (Wong et al. Centrifugal processing of cell debris and inclusion bodies from recombinant E. coli, Bioseparation 6: 361-372, 1997), report on an improved separation efficiency of the unwanted cell debris in the product from inclusion bodies on use of a disk separator when the size of the cell debris is reduced when inclusion body size is constant. A reduction of the cell debris size was achieved in this case by increasing the number of homogenizer passages from 2 to 10. By generating smaller cell debris which was preferentially separated off in the clear running of the separator, the purity of the inclusion body concentrate was increased by 58%.

If the disrupted cell suspension comprises a dissolved product of value, however, separating off the cell debris via filtration methods can be made more difficult by smaller cell debris (Storhas, 2003).

The methods described in the prior art require high amounts of energy, require many repetitions of the disruption step, are unsatisfactory with respect to the quantitative yield of functional protein, sometimes use toxic substances and are frequently accompanied with strong levels of contamination with nucleic acids which make further workup more difficult. Frequently, the methods, for cost reasons, are only applicable on a small scale, so that industrial production of proteins cannot be carried out profitably in this way.

The object of the present invention therefore was to provide a novel process for disrupting biological cells, which method successfully achieves particularly good isolation of proteins from production cells and which avoids the abovementioned disadvantages of the known methods.

The object was achieved by a method for the disruption of biological cells which successfully achieves particularly good isolation of proteins from production cells by means of a homogenizer device which

a) comprises an orifice plate having at least one inlet nozzle and an orifice plate having at least one exit nozzle, wherein, in the intermediate space between the orifice plates, a static mixer is situated and, if appropriate, mechanical energy is additionally introduced or

b) comprises an orifice plate having at least one inlet nozzle and a baffle plate, wherein, in the intermediate space between the orifice plate and the baffle plate, if appropriate a static mixer is situated and/or mechanical energy is introduced.

The invention further relates to a method of isolating a protein from a production cell by means of a homogenizer device which
a) comprises an orifice plate having at least one inlet nozzle and an orifice plate having at least one exit nozzle, wherein, in the intermediate space between the orifice plates, a static mixer is situated and, if appropriate, mechanical energy is additionally introduced or
b) comprises an orifice plate having at least one inlet nozzle and a baffle plate, wherein, in the intermediate space between the orifice plate and the baffle plate, if appropriate a static mixer is situated and/or mechanical energy is introduced.

The present invention likewise relates to a device for the disruption of biological cells which successfully achieves particularly good isolation of proteins from production cells which
a) comprises an orifice plate having at least one inlet nozzle and an orifice plate having at least one exit nozzle, wherein, in the intermediate space between the orifice plates, a static mixer is situated and, if appropriate, mechanical energy is additionally introduced or
b) comprises an orifice plate having at least one inlet nozzle and a baffle plate, wherein, in the intermediate space between the orifice plate and the baffle plate, if appropriate a static mixer is situated and/or mechanical energy is introduced.

By means of the method according to the invention, all types of biological cells can be disrupted, and, in particular proteins, subsequently to the cell disruption, can be particularly readily isolated from production cells. The method according to the invention achieves a comparable degree of cell disruption at a significantly lower differential pressure (approximately by a factor of 2) compared with the prior art. In other words, at the same differential pressure, a higher degree of cell disruption is achieved compared with the prior art. The expenditure required for subsequent killing of the biological cells by, e.g., continuous sterilization or addition of chemicals, can therefore be significantly reduced. In addition, the properties of the cell fragments are affected in such a manner that subsequent separation of the cell fragments via a separator or nozzle separator is significantly facilitated. The method is preferably used for cell disruption when subsequently nonenzymatic proteins must be isolated, in particular when surface-active proteins must be isolated. The subsequent isolation of proteins of microbial, plant origin, in particular of proteins of the class of hydrophobins, is particularly readily successfully achieved.

Hydrophobins are small proteins of about 100 amino acids which are characteristic of filamentous fungi and do not occur in other organisms. Recently, hydrophobin-like proteins have been discovered in Streptomyces coelicolor which are designated “chaplins” and likewise have highly surface-active properties. Chaplins can assemble to give amyloid-like fibrils at water/air interfaces (Classen et al. 2003 Genes Dev 1714-1726; Elliot et al. 2003, Genes Dev. 17, 1727-1740).

Hydrophobins are distributed in a water-insoluble form on the surface of various fungal structures such as, e.g., aerial hyphae, spores, fruiting bodies. The genes for hydrophobins have been isolated from Ascomycetes, Deuteromycetes and Basidiomycetes. Some fungi comprise more than one hydrophobin gene, e.g. Schizophyllum commune, Coprinus cinereus, Aspergillus nidulans. Apparently, various hydrophobins are involved in different stages of fungal development. The hydrophobins in this case are thought to be responsible for different functions (van Wetter et al., 2000, Mol. Microbiol., 36, 201-210; Kershaw et al. 1998, Fungal Genet. Biol, 1998, 23, 18-33).

As a biological function for hydrophobins, in addition to reduction of the surface tension of water for generating aerial hyphae, hydrophobicizing spores is also described (Wösten et al. 1999, Curr. Biol., 19, 1985-88; Bell et al. 1992, Genes Dev., 6, 2382-2394). In addition, hydrophobins serve for lining gas channels in fruiting bodies of lichen and as components in the recognition system of plant surfaces by fungal pathogens (Lugones et al. 1999, Mycol. Res., 103, 635-640; Hamer & Talbot 1998, Curr. Opinion Microbiol., volume 1, 693-697).

The method according to the invention may also be employed with very great success to the isolation of fusion proteins. These are taken to mean proteins which have at least one polypeptide chain which does not occur in this form in nature and has been artificially assembled from two parts. In particular, the method according to the invention is suitable for the isolation of hydrophobins.

Particularly highly suitable hydrophobins for the method according to the invention are polypeptides of the general structural formula (I)

Xₙ-C¹-X₁₋₅₀-C²-X₀₋₅-C³-Xₚ-C⁴-X₁₋₁₀₀-C⁵-X₀₋₅₀-C⁶-X₀₋₅-C⁷-X₁₋₅₀-C⁸-Xₘ (I)

wherein X can be any of the 20 naturally occurring amino acids (Phe, Leu, Ser, Tyr, Cys, Trp, Pro, His, Gln, Arg, Ile Met, Thr, Asn, Lys, Val, Ala, Asp, Glu, Gly) and the indices at X denote the number of amino acids wherein the indices n and m are numbers between 0 and 500, preferably between 15 and 300, p is a number between 1 and 250, preferably 1-100, and C is cysteine, alanine, serine, glycine, methionine or threonine, wherein at least four of the moieties named by C are cysteine, with the proviso that at least one of the peptide sequences abbreviated by Xₙ or Xₘ or Xₚ is a peptide sequence at least 20 amino acids long which is not naturally linked to a hydrophobin,
which, after coating a glass surface give a change in contact angle of at least 20°.

The amino acids named by C¹ to C⁸ are preferably cysteines; however, they can also be replaced by other amino acids of similar spatial filling, preferably by alanine, serine, threonine, methionine or glycine. However, at least four, preferably at least 5, particularly preferably at least 6, and in particular at least 7, of the positions C¹ to C⁸ should comprise cysteines. Cysteines, in the proteins according to the invention, can either be present in reduced form or form disulfide bridges with one another. Particular preference is given to the intramolecular formation of C-C bridges, in particular those having at least one, preferably 2, particularly preferably 3, and very particularly preferably 4, intramolecular disulfide bridges. In the abovedescribed replacement of cysteines by amino acids of similar space filling, advantageously, those C positions are replaced in pairs which can form intramolecular disulfide bridges with one another.

If, in the positions designated by X, cysteines, serines, alanines, glycines, methionines or threonines are also used, the numbering of the individual C positions in the general formulae can change correspondingly. Particularly advantageous polypeptides are those of the general formula (II)

Xₙ-C¹-X₃₋₂₅-C²-X₀₋₂-C³-X₅₋₅₀-C⁴-X₂₋₃₅-C⁵-X₂₋₁₅-C⁶-X₀₋₂-C⁷-X₃₋₃₅-C⁸-Xₘ (II)

wherein X can be any of the 20 naturally occurring amino acids (Phe, Leu, Ser, Tyr, Cys, Trp, Pro, His, Gin, Arg, Ile Met, Thr, Asn, Lys, Val, Ala, Asp, Glu, Gly) and the indices at X are the number of amino acids, wherein the indices n and m are numbers between 2 and 300 and C is cysteine, alanine, serine, glycine, methionine or threonine, wherein at least four of the moieties named by C are cysteine, with the proviso that at least one of the peptide sequences abbreviated by Xₙ or Xₘ is a peptide sequence at least 35 amino acids long which is not naturally linked to a hydrophobin, which, after coating a glass surface, cause a change in contact angle of at least 20°.

The origin of the hydrophobins is of no importance in this case. For instance, the hydrophobins can have been isolated, for example, from microorganisms such as, e.g., bacteria, yeast and fungi. In particular hydrophobins which have been obtained by means of genetically modified organisms come into consideration according to the invention.

Using the method according to the invention, proteins may be more readily isolated. Isolation from a production cell is usually one of the first steps in purification of a protein when the protein is produced and stored intracellularly.

A cell is designated as a production cell in this case which is any type of cell or cell assembly, in particular those cells of animal, plant or fungal origin or microorganisms from the group of bacteria or Archaea. Preferred production cells are recombinant organisms. Particularly highly suitable production cells are prokaryotes (including the Archaea) or eukaryotes, particularly bacteria including halobacteria and methanococcae, fungi, insect cells, plant cells and mammalian cells, particularly preferably, Escherichia coli, Bacillus subtilis, Bacillus. megaterium, Aspergillus oryzea, Aspergillus nidulans, Aspergillus niger, Pichia pastoris, Pseudomonas spec., Lactobacillen, Hansenula polymorpha, Trichoderma reesei, SF9 (and/or related cells), CHO.

The production cell can be used directly after culture (e.g. fermentation) in the method according to the invention; however, it is also possible first to kill the production cell, for example by sterilization, and if appropriate to enrich the cell mass by filtration of the culture medium.

The homogenizer device for cell disruption either comprises an orifice plate having at least one inlet nozzle and an orifice plate having at least one exit nozzle, wherein the nozzles are arranged axially to one another. In the intermediate space between the orifice plates there is situated a static mixer. If appropriate, in addition, mechanical energy is introduced.

The orifice plates usable by the method according to the invention have at least one orifice, i.e. at least one nozzle. In this case the two orifice plates can each have any desired number of orifices, but preferably no more than 5 orifices each, particularly preferably no more than three orifices each, very particularly preferably no more than two orifices each, and in particular preferably no more than one orifice each. Both orifice plates can have a different number or the same number of orifices, but preferably both orifice plates have the same number of orifices. Generally, the orifice plates are perforated plates having at least one orifice each.

In another embodiment of this method according to the invention, the second orifice plate is replaced by a sieve, i.e. the second orifice plate has a multiplicity of orifices or nozzles. The sieves which are usable can extend over a large range of pore sizes, generally the pore sizes are between 0.1 and 250 μm, preferably between 0.2 and 200 μm, particularly preferably between 0.3 and 150 μm, and in particular between 0.5 and 100 μm.

The orifices or nozzles can have any conceivable geometric shape, they can, for example, be circular, oval, angular having any desired number of corners which, if appropriate, can also be rounded, or else star-shaped. Preferably, the orifices have a circular shape.

The orifices generally have a diameter of 0.05 mm to 1 cm, preferably 0.08 mm to 0.8 mm, particularly preferably 0.1 to 0.5 mm, and in particular 0.2 to 0.4 mm.

The two orifice plates are preferably constructed in such a manner that the orifices or nozzles are arranged axially to one another. Axial arrangement is taken to mean that the direction of flow generated by the geometry of the nozzle orifice is identical in the case of both orifice plates. The orifice orientations of the inlet nozzle and outlet nozzle need not for this lie on a line, they can also be offset in parallel, as proceeds from the above considerations. Preferably, the orifice plates are orientated in parallel.

However, other geometries, in particular non-parallel orifice plates, or different orifice orientations of the inlet and outlet nozzles are possible.

The thickness of the orifice plates can be as desired. Preferably, the orifice plates have a thickness in the range 0.1 to 100 mm, preferably 0.5 to 30 mm, and particularly preferably 1 to 10 mm. In this case the thickness (l) of the orifice plates is selected such that the quotient of the diameter (d) of the orifices and thickness (l) is in the range of 1:1, preferably 1:1.5, and particularly preferably 1:2.

The intermediate space between the two orifice plates can be any desired length, generally the length of the intermediate space is 1 to 500 mm, preferably 10 to 300 mm, and particularly preferably 20 to 100 mm.

In the intermediate space between the orifice plates, there is situated according to the invention a static mixer which can fill, wholly or in part, the section between the two orifice plates. Preferably, the static mixer extends over the entire length of the intermediate space between the two orifice plates. Static mixers are known to those skilled in the art. It can be in this case, for example, a valve mixer or a static mixer having bore holes, one made of fluted lamellae, or one made of interdigitating ridges. In addition, it can be a static mixer in spiral shape or in N shape or one having heatable or coolable mixing elements.

By installing a static mixer into the intermediate space between the two orifice plates, the stability of the resultant protein suspension is considerably improved.

In addition to the static mixer, in the intermediate space between the two orifice plates, mechanical energy can furthermore be introduced. The energy can be introduced, for example, in the form of mechanical vibrations, ultrasound or rotational energy. By this means, a turbulent flow is generated, the effect of which is that the particles in the intermediate space do not agglomerate.

Alternatively to this first variant, the mixing device can comprise an orifice plate having at least one inlet nozzle and a baffle plate, wherein, in the intermediate space between the orifice plate and the baffle plate, if appropriate, a static mixer is situated. Alternatively to, or in addition to, the static mixer, mechanical energy can be introduced in the intermediate space.

The aforesaid applies to the orifice plate having inlet nozzle, the intermediate space having a static mixer and mechanical energy introduction.

In this variant, the second orifice plate is replaced by a baffle plate. The baffle plate generally has a diameter which is 0.5 to 20%, preferably 1 to 10%, smaller than the tube diameter at the position at which the baffle plate is installed.

In general, the baffle plate can have any geometrical shape, preferably be in the shape of a round disk, so that in the frontal view a ring gap may be seen. For example, the shape of a slot or a channel is also conceivable.

The baffle plate can, similarly to the second orifice plate in the abovedescribed variant, be mounted at different distances to the first orifice plate. The intermediate space between the orifice plate and the baffle plate is thereby as long as desired, generally the length of the intermediate space is 1 to 500 mm, preferably 10 to 300 mm, and particularly preferably 20 to 100 mm.

The method according to the invention, compared with the methods known from the prior art, has some advantages since particularly high yields of the protein in active form are obtained.

The temperature at which the biological cell disruption proceeds by the method according to the invention is generally 0 to 150° C., preferably 5 to 80° C., particularly preferably 20 to 40° C. In this case, all of the homogenizer units used in the device can be heated or cooled.

The homogenization is generally carried out at pressures above atmospheric pressure, i.e. >1 bar. In this case, however, the pressures do not exceed a value of 10 000 bar, such that preferably homogenization pressures of >1 bar to 10 000 bar, preferably 5 to 2000 bar, and particularly preferably 10 to 1500 bar, are set.

The production cell concentrations used in the method according to the invention (as total dry matter) are about 3 to 25% by weight, preferably 5-15% by weight.

The protein isolates obtained by the method according to the invention can, depending on the intended use, be used either directly or after renaturing the protein, further purification and if appropriate formulation.

The invention further relates therefore to a production method for a protein which, inter alia, comprises the abovedescribed isolation from a production cell.

The proteins can be purified using known methods subsequently to the method according to the invention. Purification succeeds particularly readily when the cell protein is present in what are termed inclusion bodies. In this case, these may be particularly advantageously separated off selectively from the unwanted cell debris by centrifugal separation in a separator or nozzle separator. Subsequently to the renaturation of the protein, further purification can be achieved using known chromatographic methods such as molecular sieve chromatography (gel filtration), such as Q-Sepharose chromatography, ion-exchange chromatography and hydrophobic chromatography, and also using other conventional methods such as centrifugal separation, ultrafiltration, crystallization, salting out, dialysis and native gel electrophoresis. On production of a soluble cell protein, subsequently to the cell disruption, directly with known chromatographic methods such as Q-Sepharose chromatography, ion-exchange chromatography and hydrophobic chromatography, and also with other conventional methods such as centrifugal separation, ultrafiltration, crystallization, salting out, dialysis and native gel electrophoresis proceed. Suitable methods are described, for example, in Cooper, F. G., Biochemische Arbeitsmethoden [Biochemical working methods], Verlag Water de Gruyter, Berlin, New York, or in Scopes, R., Protein Purification, Springer Verlag, New York, Heidelberg, Berlin.

Subsequently, or alternatively, if desired, the protein can be formulated by drying and, if appropriate, addition of aids and preservatives.

### EXPERIMENTAL PART

### Fermentation and Workup (Examples 1-8)

200 ml of complex medium are inoculated in a 1000 ml conical flask having two side chicanes with an E. coli strain of LB-Amp plate (100 μg/ml of ampicillin) (=first preculture). The strain is incubated to an OD_{600 nm} of approximately 3.5 at 37° C. on a shaker having dₒ=2.5 cm at 200 rpm. Subsequently, 4 further 1000 ml conical flasks having chicanes (each having 200 ml of complex medium) are each inoculated with 1 ml of the first preculture and incubated at 37° C. in the shaking cabinet (dₒ=2.5 cm, n=200 rpm) (=2nd preculture). As soon as the OD_{600 nm} is >6, the prefermenter filled with complex medium is inoculated from this second shake culture. After reaching an OD_{600 nm}>9 or OTR=80 mmol/(l-h), the main fermenter is inoculated. The main culture is run to an OD of 70 in the fed-batch culture. The fermentation is ended by cooling to 4° C. The cell mass is run through a disk separator at a flow rate of 200 l/h. 3300 kg of fermentation broth produce 860 kg of concentrate. The resuspended cells are made up with 1200 kg of water and the viable cell count is determined (=null sample). The dry matter content (DM) of the resuspended cell broth is 5% by weight.

For the cell disruption, the suspended cells are brought to the desired operating pressure by a high-pressure pump (FIG. 1). The subsequent pressure expansion takes place in the described high-pressure orifice plate. In this process the cell disruption takes place. Shearing, extension and turbulent flow forces are responsible which attack the suspended microorganisms. On a laboratory scale, a flow rate of approximately 25 l/h and an operating pressure downstream of the pump of up to 2500 bar are employed. The temperature on the intake side of the pump is optional (usually room temperature or precooled to 4° C.). On the outlet side the dissipated energy leads to a temperature elevation of up to 50° C. Therefore, a heat exchanger is installed downstream of the high-pressure nozzle. In addition, the nozzle itself can be/is already cooled. The following nozzle pairs are used for the cell disruption:

500 bar: 0.2 and 0.4 mm

1000-2400 bar 0.1 and 0.2 mm

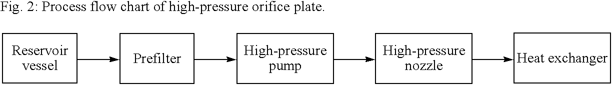

Fermentation and Workup (Example 9)

200 ml of complex medium are inoculated in a 1000 ml conical flask having two side chicanes with a YaaD-DewA-His6-expressing E. coli strain of LB-Amp plate (100 μg/ml of ampicillin) (=first preculture). The strain is incubated to an OD_{600 nm} of approximately 3.5 at 37° C. on a shaker having dₒ=2.5 cm at 200 rpm. Subsequently, 4 further 1000 ml conical flasks having chicanes (each having 200 ml of complex medium) are each inoculated with 1 ml of the first preculture and incubated at 37° C. in the shaking cabinet (dₒ=2.5 cm, n=200 rpm) (=2nd preculture). As soon as the OD_{600 nm} is >6, the prefermenter filled with complex medium is inoculated from this second shake culture. After an OD_{600 nm} >9 or OTR=80 mmol/(l-h) is reached, the main fermenter is inoculated. The main culture is run in the fed-batch method in mineral medium. At an OD_{600 nm}>70, the cells are induced with 50 μm of IPTG. After an induction time between 4 and 20 h, the fermentation is interrupted and the vessel content is cooled to 4° C. The cells, subsequently to the fermentation, are separated off from the fermentation broth by means of a nozzle separator having a flow rate of 700 l/h (concentrate: DM=13.4% by weight, biological DM=10.4% by weight) and resuspended in deionized water. After the cells are separated again by using the nozzle separator, the dry matter content is 14.9% by weight, and biological dry matter is 13.3% by weight.

The scale up from laboratory scale (examples 1-8) to industrial scale was carried out at throughputs of up to 700 l/h and pressures up to 2500 bar. For temperatures on the input side, the same conditions apply as in examples 1-8 (see above). The cell-containing suspension is passed by means of a compressed air membrane pump through a 70 μm prefilter. At an inlet pressure of at least 1.5 bar, the cell-containing suspension is brought to the appropriate pressure using a high-pressure piston pump and then passed through what is termed the orifice plate block. The orifice plate block comprises 2 orifice plates. The first orifice plate has 14 bore holes each having a diameter of 0.1 or 0.2 mm (see above). After passage through a bore hole having 8 mm diameter, the cell-containing suspension passes through the second orifice plate having a diameter of 1.5 mm to the unpressurized side. Subsequently, the cell-containing suspension passes through the cooler.

Determination of Viable Cell Count

For determination of the viable cell count, in each case 100; 10 and 1 μl of suspension are spread on LB AMP100 agar plates which are incubated overnight at 37° C. The colony forming units (CFU) are subsequently enumerated and the viable cell count per unit volume is estimated.

Definitions

| | |
|---|---|
| Total dry matter: | Dried sample, comprises all of the dry substance |
| Biological dry matter: | Sample washed twice and subsequently dried |

Degree of Disruption

The degree of disruption (A) is defined from

$$A = \frac{{N}_{o} - N}{{N}_{o}}$$

Nₒ Viable cell count prior to cell disruption

N Viable cell count after cell disruption (single passage)

Activity Test

The coating properties of the redissolved spray-dried or spray-granulated hydrophobin fusion protein are used for evaluation of the protein activity. The coating properties are preferably evaluated on glass and Teflon as models of hydrophilic and hydrophobic surfaces, respectively.

Glass:
concentration of hydrophobin: 50 mg/l
incubation of glass slides overnight (temperature: 80° C.) in 10 mM Tris pH 8
after coating, washing in deionized water
thereafter incubation for 10 min/80° C./1% SDS
washing in deionized water

Teflon:
concentration: 50 mg/l
incubation of Teflon slides overnight (temperature: 80° C.) in 10 mM Tris pH 8
after coating washing in deionized water
thereafter incubation for 10 min/80° C./1% SDS
washing in deionized water

The samples are dried in air and the contact angle (in degrees) of a drop of 5 μl water is determined. This gives the following results, for example:

Batch having YaaD-DewA fusion protein (control: without protein; Yaab-DewA-His₆: 100 mg/l purified fusion partner):

after 1% SDS 80° C.

| | Teflon | Glass |
|---|---|---|
| Control | 96.8 | 30 |
| YaaD | 97.4 | 38.7 |
| 50 mg/L | 85.9 | 77.9 |

### Example 1

One part of the resuspended cells is disrupted via a Microfluidizer Processor M-7125-30 S.N. 200414 using the digestion chambers “Ceramic IXC H10Z-6 slot” and “Ceramic APMH30Z” from Microfluidics at a differential pressure of 1750 bar. The flow rate in this case is 3.3 l/min. The viable cell count which results in the cell disruptate after a single passage is listed in table 1.

### Example 2

One part of the resuspended cells is disrupted via the high-pressure orifice plate (HD orifice plate) at a differential pressure of 500 bar. The flow rate in this case is 3.3 l/min. The viable cell count which results in the cell disruptate after a single passage is listed in table 1.

### Example 3

One part of the resuspended cells is disrupted via the high-pressure orifice plate (HD orifice plate) at a differential pressure of 1000 bar. The flow rate in this case is 3.3 l/min. The viable cell count which results in the cell disruptate after a single passage is listed in table 1.

### Example 4

One part of the resuspended cells is disrupted by the high-pressure orifice plate (HD orifice plate) at a differential pressure of 1200 bar. The flow rate in this case is 3.3 l/min. The viable cell count which results in the cell disruptate after a single passage is listed in table 1.

### Example 5

One part of the resuspended cells is disrupted via the high-pressure orifice plate (HD orifice plate) at a differential pressure of 1400 bar. The flow rate in this case is 3.3 l/min. The viable cell count which results in the cell disruptate after a single passage is listed in table 1.

### Example 6

One part of the resuspended cells is disrupted via the high-pressure orifice plate (HD orifice plate) at a differential pressure of 1800 bar. The flow rate in this case is 3.3 l/min. The viable cell count which results in the cell disruptate after a single passage is listed in table 1.

### Example 7

One part of the resuspended cells is disrupted via the high-pressure orifice plate (HD orifice plate) at a differential pressure of 2000 bar. The flow rate in this case is 3.3 l/min. The viable cell count which results in the cell digest after a single passage, is listed in table 1.

### Example 8

One part of the resuspended cells is disrupted via the high-pressure orifice plate (HD orifice plate) at a differential pressure of 2400 bar. The flow rates in this case are 3.3 l/min. The viable cell count which results in the cell disruptate after a single passage is listed in table 1.

The examples show (see also FIGS. 2 and 3) that the high-pressure orifice plate, at the same differential pressure, leads to a significantly higher degree of disruption compared with the prior art.

### Example 9

The cell suspension is disrupted using the high-pressure orifice plate at a differential pressure of 1000; 1500 and 2000 bar at a flow rate of 700 l/h and the respective viable cell count determined (see table 2). The cell broth disrupted at 2000 bar differential pressure is run through a nozzle separator at a flow rate of 400 l/h. The sought-after inclusion bodies preferentially accumulate in concentrate 1 (DM=14.8% by weight, m=347 kg), whereas the cell debris is preferentially separated off with the clear running 1 (DM=8.2% by weight, m=508 kg). The concentrate is made up with 500 l of water and again run through the nozzle separator at 400 l/h (=wash step). The sought-after inclusion bodies preferentially accumulate in concentrate 2 (DM=11 % by weight, m=343 kg), whereas the cell debris is preferentially separated off with the clear running 2 (DM=2.9% by weight, m=508 kg). The wash step is repeated. This results in an IB yield of 370 kg having 7.9% by weight dry matter content. This resultant concentrate is set to pH 12.5 and, after 15 min, the pH is lowered to 9. The neutralized hydrophobin-comprising solution is run through a tube centrifuge for solids separation. According to SDS-PAGE analysis, the hydrophobin, after the concluding centrifugation is present in the supernatant. This supernatant is hereinafter termed “aqueous hydrophobin solution”. The dry matter content of the aqueous hydrophobin solution is 3,4% by weight. Mannitol is added to the hydrophobin-comprising solution as drying aid at a ratio DM:mannitol=1:1. This solution is sprayed cocurrently in 1200 kg/h of nitrogen using a two-fluid nozzle of the Geyrig Gr.0 type at an injection rate of 41 kg/h.

The spraying tower has a diameter of 800 mm and a height of 12 m. The inlet temperature of the drying gas in this case is 161 DEG. The exit temperature of the drying gas is 80 DEG. The separation proceeds in the filter in which 31.7 kg of dry material are recovered. 6.1 kg of dry material are cleaned out of the tower. The contact angles resulting from the activity test of the redissolved hydrophobin-comprising dry material are listed in table 2. The protein gel of the redissolved dry material is shown in FIG. 3.

TABLE 1
Viable cell count and degree of disruption
for the null sample and examples 1-8.

| | | Differential | Viable cell | Degree of |
|---|---|---|---|---|
| | | pressure | count | disruption |
| Example | Apparatus | [bar] | [1/ml] | [-] |
| Null sample | — | — | 1.2*10⁹ | — |
| Example 1 | Microfluidics | 1750 | 3.2*10⁷ | 0.973 |
| Example 2 | HD-orifice plate | 500 | 3.8*10⁸ | 0.683 |
| Example 3 | HD-orifice plate | 1000 | 5.2*10⁶ | 0.996 |
| Example 4 | HD-orifice plate | 1200 | 5.1*10⁶ | 0.996 |
| Example 5 | HD-orifice plate | 1400 | 1.3*10⁶ | 0.999 |
| Example 6 | HD-orifice plate | 1800 | 2*10⁵ | 1 |
| Example 7 | HD-orifice plate | 2000 | 1*10⁵ | 1 |
| Example 8 | HD-orifice plate | 2400 | 2*10⁴ | 1 |

TABLE 2
Viable cell count and degree of disruption for example 9.

| | | Differential | Viable cell | Degree of |
|---|---|---|---|---|
| | | pressure | count | disruption |
| Example | Apparatus | [bar] | [1/mL] | [-] |
| Null sample | — | — | 1.3*10⁹ | — |
| Example 9.1 | HD-orifice plate | 1000 | 5.5*10⁷ | 0.958 |
| Example 9.2 | HD-orifice plate | 1500 | 5.8*10⁷ | 0.955 |
| Example 9.3 | HD-orifice plate | 2000 | 3*10⁶ | 0.998 |

TABLE 3
Contact angle after spray drying hydrophobin A with mannitol.

| | Glass | Teflon |
|---|---|---|
| Control | 20.5 | 108.2 |
| Example 9 | 66.2 | 85.5 |

### EXPLANATION OF THE FIGURES

FIG. 1: Viable cell count [1/ml] of examples 1-8.

FIG. 2: Degree of disruption [-] of examples 1-8.

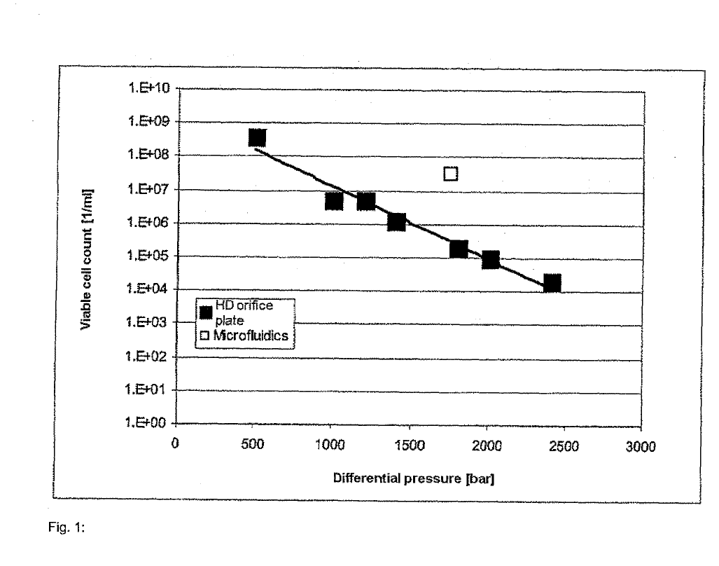

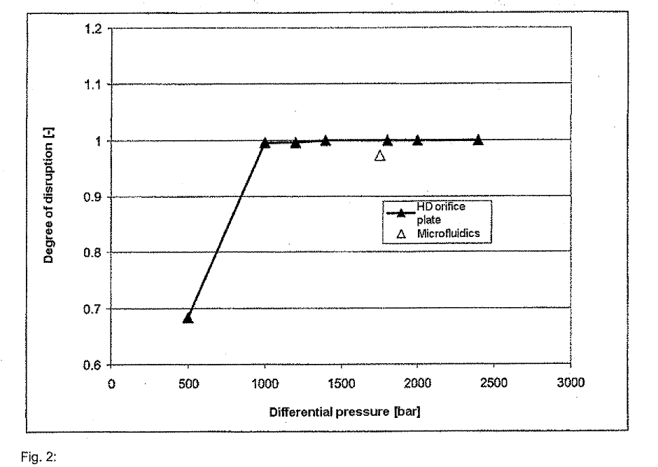

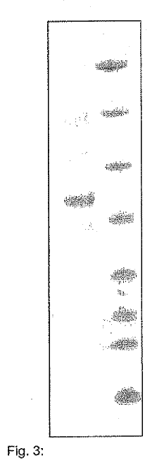

FIG. 3: Protein gel example 9:
4-12% Bis-Tris Gel/MES buffer, left: after spray drying hydrophobin A with mannitol, right: Marker: Prestained SDS-Page Standards, application/slot: 15 μg Pr

1. A method for the disruption of biological cells by means of a homogenizer device which
a) comprises an orifice plate having at least one inlet nozzle and an orifice plate having at least one exit nozzle, wherein, in the intermediate space between the orifice plates, a static mixer is situated and, if appropriate, mechanical energy is additionally introduced or
b) comprises an orifice plate having at least one inlet nozzle and a baffle plate, wherein, in the intermediate space between the orifice plate and the baffle plate, if appropriate a static mixer is situated and/or mechanical energy is introduced.

2. The method according to claim 1, wherein the cells are a natural or recombinant organism.

3. A method of isolating a protein from a production cell by means of a homogenizer device which
a) comprises an orifice plate having at least one inlet nozzle and an orifice plate having at least one exit nozzle, wherein, in the intermediate space between the orifice plates, a static mixer is situated and, if appropriate, mechanical energy is additionally introduced or
b) comprises an orifice plate having at least one inlet nozzle and a baffle plate, wherein, in the intermediate space between the orifice plate and the baffle plate, if appropriate a static mixer is situated and/or mechanical energy is introduced.

4. The method according to claim 3, wherein the protein is a hydrophobin.

5. The method according to claim 1, wherein the production cells are killed before homogenization.

6. A method of producing a recombinant protein comprising a method step according to claim 1.
